(19) United States
(12) Patent Application Publication
(10) Pub. No.: US 2010/0212673 A1
(43) Pub. Date: Aug. 26, 2010

(54) Biohazardous spray shield

(76) Inventor: Kaytherine C. Shumpert, Fulton, MS (US)

Correspondence Address: Kaytherine C. Shumpert, 2101 Martin L. King Rd., Fulton, MS, 38843, US

(21) Appl. No.: 12/378,732

(22) Filed: Feb. 20, 2009

(51) Int. Cl. A61F 13/00

(52) U.S. Cl. 128/846

(57) ABSTRACT

The Biohazardous Spray Shield is a product designed to shield healthcare workers and others from accidental contact with body fluids or other hazardous material. The wrist mounted shield acts as a clear barrier that blocks most spittle or body fluids that may contaminate personnel. The Biohazardous Spray Shield is easily mounted on the wrist, secured with Velcro straps. The plastic shield slides into the grooves on the plastic block, instantly placing a barrier between the individual and a potential hazard.

### GENERAL DESCRIPTION

A wristband-mounted, clear plastic guard.

### APPLICATION

Provides a barrier between the patient and the health care worker.

For example, when swabbing a patient's throat, the health care worker can view

the throat through the clear, plastic guard and avoid the spray or spittle that often

accompanies swabbing.

### DETAILED DESCRIPTION

Wristband: Two 14 cm×2 cm straps connected to opposite sides of a 4 cm (w)×4 cm (l)×2 cm (h) formed plastic block guardholder.

Strap #1 has female Velcro on the top side of the strap, reaching 7 cm up the strap from the end.

Strap #2 has male Velcro on the underside of the strap, reaching 7 cm up the strap from the end.

Guardholder: Contains three 3 cm deep grooves across the width of the holder. #1 groove is angled at 60 degrees to the left. #2 groove is vertical. #3 groove is angled at 60 degrees to the right.

Shield: Clear Loncet plastic 22 cm (l)×15 cm (h)

Assembly: Place wristband on wrist, fasten with the Velcro, slide Loncet plastic shield into selected groove, perform exam.

### BIOHAZARDOUS SPRAY SHIELD DRAWINGS

FIG. 1A: TOP VIEW OF STRAPS AND PLASTIC BLOCK

FIG. 1B: SIDE VIEW OF ASSEMBLED STRAPS AND BLOCK

FIG. 1C: TOP VIEW OF ASSEMBLED STRAPS AND BLOCK

FIG. 2: CLEAR LONCET SHIELD

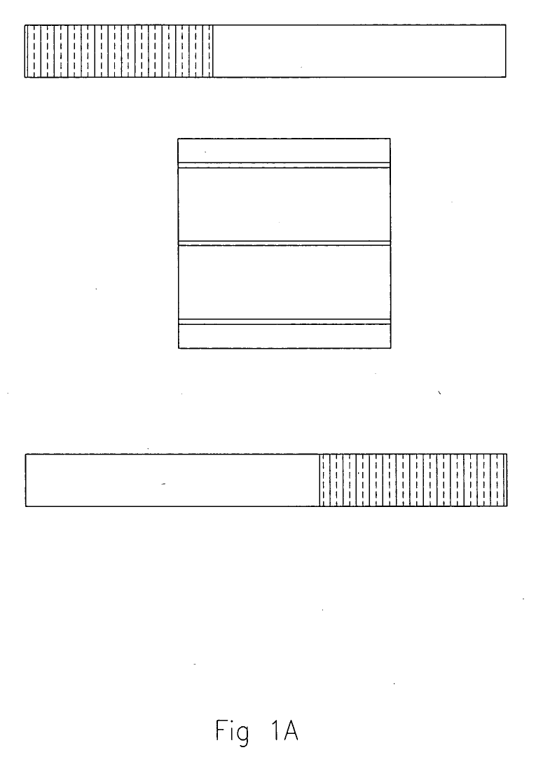

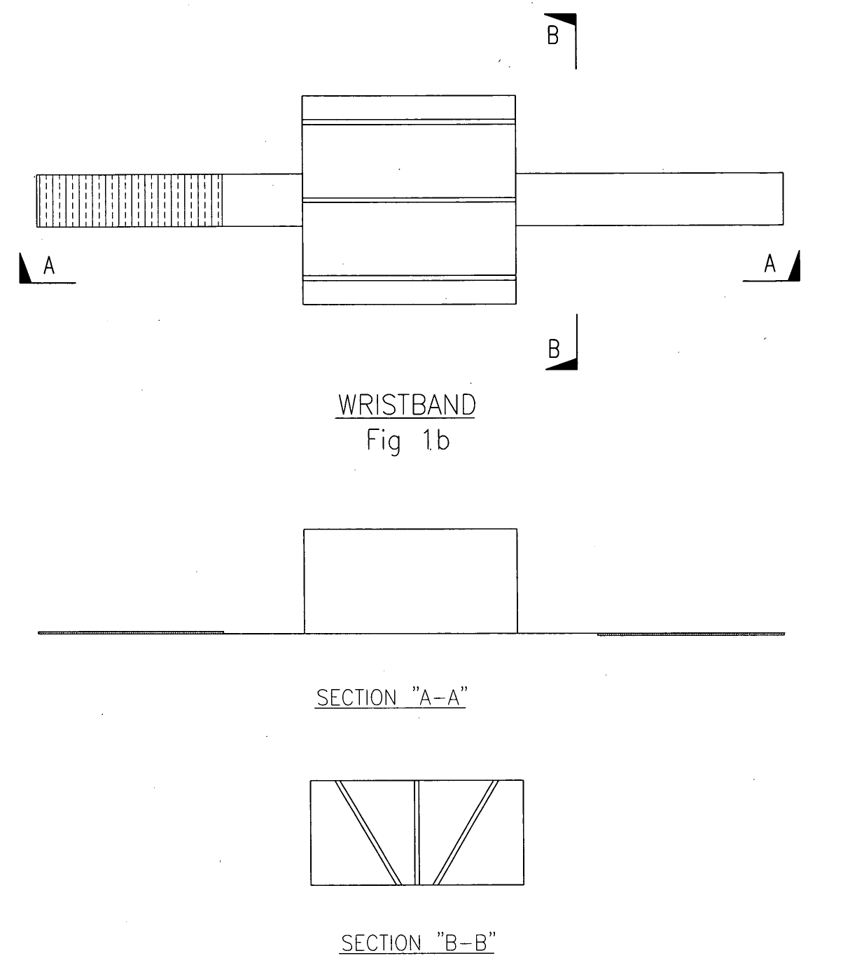

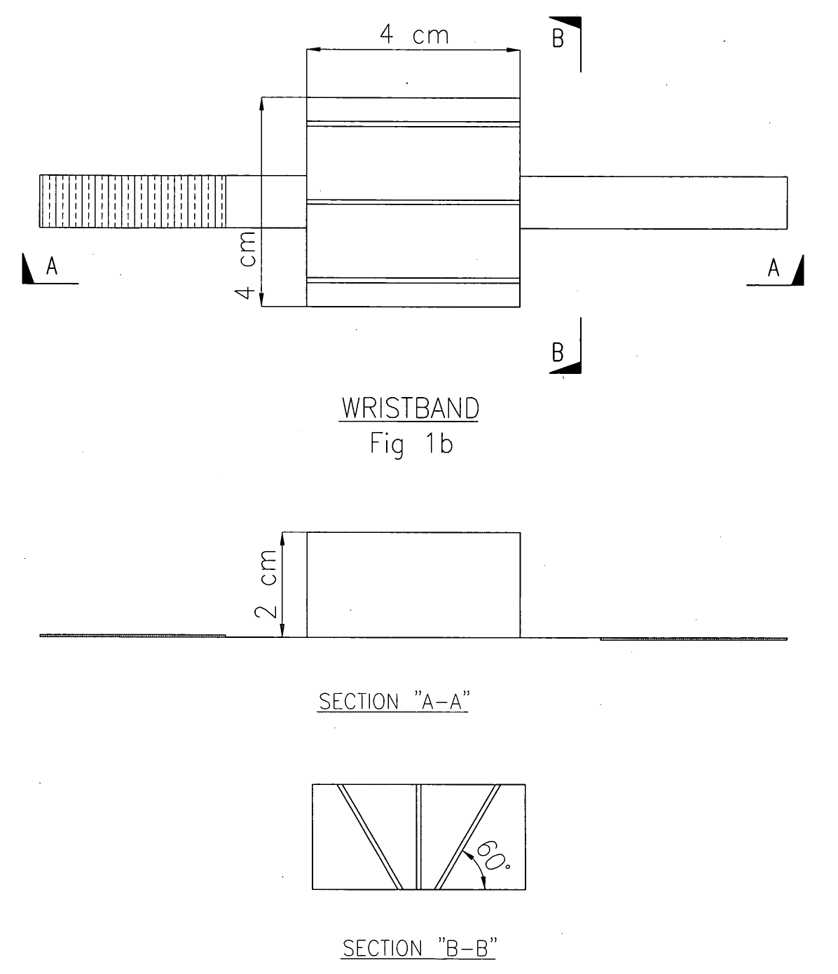

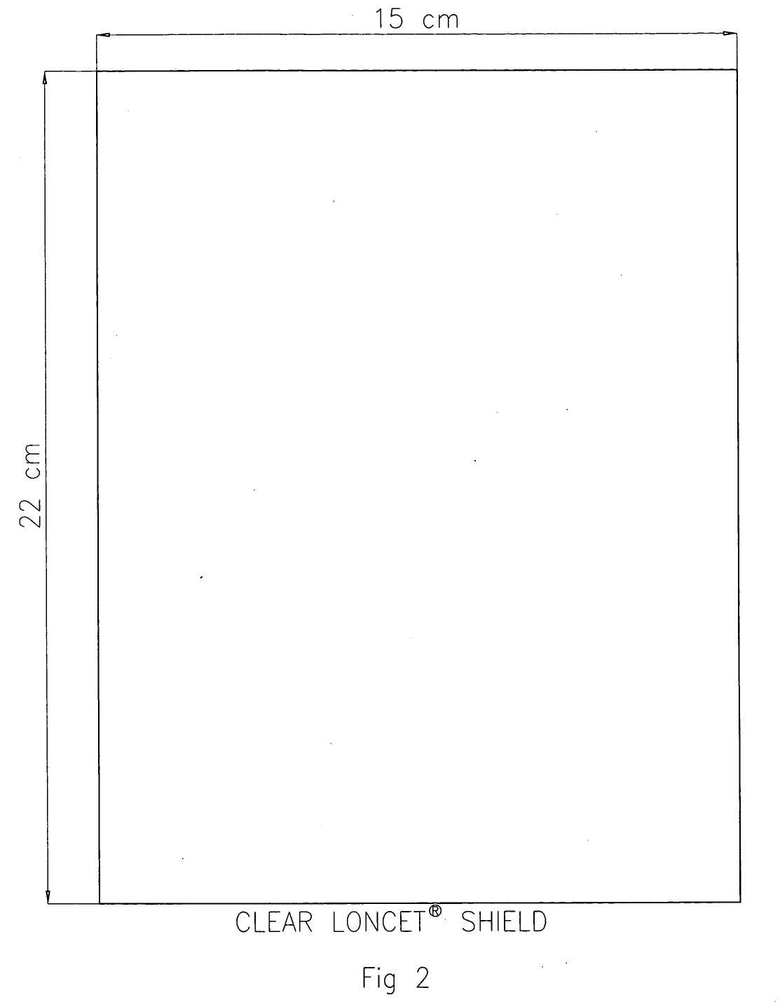

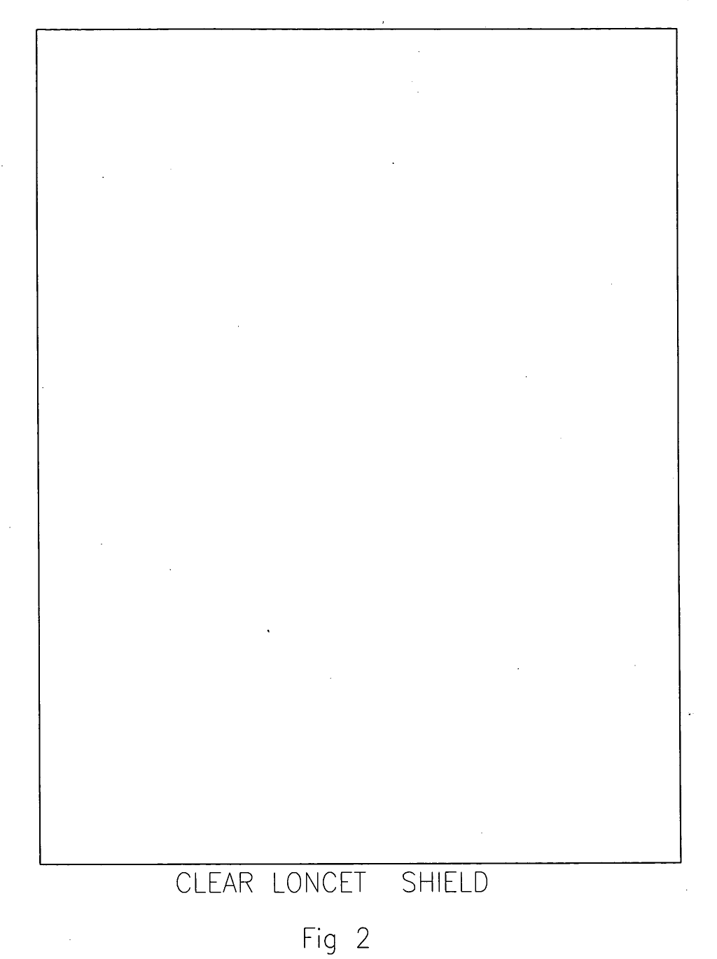

1. The Biohazardous Spray Shield will reduce contact between body fluids and medical personnel. It will effectually guard against saliva and other body fluids to reduce the number of incidental contacts of doctors, dentist, nurses, respiratory therapist, lab technicians and other health care providers who are in daily contact with the general public. The Biohazardous Spray Shield will decrease the number of staff sick days used, simply because there is a barrier that decreases the likelihood of exposure. Unlike wearing a facial shield, The Biohazardous Spray Shield does not block the vision of either the healthcare provider or the patient and more physicians would be more prone to use it on a routine oral, vaginal and rectal exams. This patent must be granted as soon as possible because any delay just increases the number of unnecessary exposures to body fluids.
